(19) United States
(12) Patent Application Publication
(10) Pub. No.: US 2005/0009861 A1
(43) Pub. Date: Jan. 13, 2005

(54) Combination use of acetylcholinesterase inhibitors and GABAa inverse agonists for the treatment of cognitive disorders

(75) Inventors: Anabella Villalobos, Niantic, CT (US); James V. Cassella, Guilford, CT (US); Lavanya Rajachandran, Wallingford, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/912,993

(22) Filed: Aug. 6, 2004

Related U.S. Application Data
(63) Continuation of application No. 09/976,347, filed on Oct. 12, 2001, now abandoned.
(60) Provisional application No. 60/241,145, filed on Oct. 17, 2000.

(51) Int. Cl. A61K 31/4745

(52) U.S. Cl. 514/300

(57) ABSTRACT

This invention provides a composition for treating a cognitive disorder, which comprises an acetylcholinesterase inhibitor, and a GABA_{A} inverse agonist selected from a compound of the formula 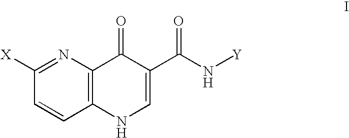 where X and Y are as defined herein.

This application claims priority from U.S. provisional application Ser. No. 60/241,145, filed Oct. 17, 2000, which is incorporated herein by reference in its entirety.

### BACKGROUND OF THE INVENTION

The present invention relates to the combination use of acetylcholinesterase (AchE) inhibitors and GABA_{A} inverse agonists, which results in cognition enhancement. Such a combination is useful in treatment of disorders associated with cognition impairment including, but not limited to, Alzheimer's disease, mild cognitive impairment, age related cognitive decline, vascular dementia, Parkinson's disease, memory impairment associated with depression or anxiety, psychosis, Down's Syndrome, stroke, traumatic brain injury and attention deficit disorder.

Alzheimer's disease (AD) is characterized by a progressive loss of memory and inability to carry out normal activities of daily living and is frequently accompanied by changes in behavior and personality. Alzheimer's disease is associated with degeneration of cholinergic neurons, which play a fundamental role in cognitive functions. It is known that acetylcholinesterase inhibitors are effective in enhancing cholinergic activity and are useful in improving memory and function in Alzheimer's Disease patients. Rogers, S. L., Friedhoff, L. T., Apter, J. T., Richter, R. W., Hartford, J. T., Walshe, T. M., Baumel, B., Linden, R. D., Kinney, F. C., Doody, R. S., Borison, R. L. and Ahem, G. L., The Efficacy and Safety of Donepezil in Patients with Alzheimer's Disease: Results of a US Multicentre, Randomized, Double-blind, Placebo-controlled Trial Dementia, 1996, volume 7, issue 6, pages 293-303. Rogers, S. L., Doody, R., Mohs, R. and Friedhoff, L. T., E2020 Produces Both Clinical Global and Cognitive Test Improvement in Patients with Mild to Moderately Severe Alzheimer's Disease: Results of a 30 week Phase III Trial, Neurology, 1996, volume 46, issue 2, Suppl. A217.

Modulators of the GABA_{A} receptors are capable of enhancing cognition in rodent models of cognition. In such models, it has been demonstrated that a selective inverse agonist profile can lead to cognitive enhancers devoid of or with minimum proconvulsant, anxiogenic and stimulant activity. The GABA_{A} inverse agonist binding and functional profile is described below:

TABLE 1

| Binding | Oocyte Functional Profile | | | |
|---|---|---|---|---|
| Ki | α1β2γ2 | α2β3γ2 | α3β3γ2 | α5β3γ2 |
| Ro15-1788 | EC₅₀/ | EC₅₀/ | EC₅₀/ | EC₅₀/ |
| Rat cortex | Efficacy | Efficacy | Efficacy | Efficacy |
| 100 nM, | 200 nM, | Any*/>10% | Any*/>10% | 200 nM, |
| preferably | preferably | | | preferably |
| <30 nM | <150 nM/ | | | <150 nM/ |
| | <−10% or >+10% | | | <−10% |

*Though a wide range of EC₅₀ values at the α2β3γ2 and α3β3γ2 subtype receptors is permitted, in practice the “Any/>10%” criteria are used for compounds having EC₅₀ values at these subtypes below or equal to 100 times the EC₅₀ values at the α1β2γ2 and α5β3γ2 subtype receptors. When the EC₅₀ value of the compound at the α2β3γ2 and α3β3γ2 subtype receptor is more than 100 times greater than at the α1β2γ2 and α5β3γ2 subtype receptors then <10% in vitro efficacy would be acceptable.

A compound is identified as having cognitive enhancing potential when the EC₅₀ value of the compound at the α1β2γ2 and/or α5β3γ2 subtype receptors is less than 200 nM, preferably less than 150 nM, and the efficacy measured is less than −5% or preferably less than −10%, and the efficacy measured at the α2β3γ2 and α3β3γ2 subtype receptors is greater than 5% or preferably greater than 10%.

The combination of a GABA_{A} cognitive enhancer and an AChE inhibitor results in greater (additive/synergistic) efficacy or cognitive/behavioral improvement in the treatment of the above disorders in comparison to the efficacy displayed by either agent alone. In addition, such a combination allows lower doses of each agent to be administered, resulting in efficacy similar to or greater than the one observed with higher doses of either agent alone, and reduced side effects (or higher therapeutic index).

### SUMMARY OF THE INVENTION

This invention provides a combination treatment of cognitive disorders in a mammal, wherein an acetylcholinesterase inhibitor and a GABA_{A} inverse agonist are administered to the mammal separately, sequentially or simultaneously so as to obtain the benefit of the combination.

More specifically, the invention provides a pharmaceutical composition comprising an acetylcholinesterase inhibitor and an inverse agonist of the GABA_{A} α5 receptor wherein the inverse agonist has a functional efficacy at the α5 receptor subtype of less than 20%, and a functional efficacy at the α₁, α₂ and α₃ receptor subtypes of between −20 and +20%, and a pharmaceutically acceptable carrier.

This invention also provides a pharmaceutical composition comprising an acetylcholinesterase inhibitor and a GABA_{A} inverse agonist wherein the inverse agonist has a functional efficacy at the α1 and/or α5 receptor subtypes of less than −5%, preferably less than −10%, and the efficacy measured at the α2 and α3 receptor subtypes is greater than 5% or preferably greater than 10%, and a pharmaceutically acceptable carrier.

This invention also provides a pharmaceutical composition comprising an acetylcholinesterase inhibitor and a GABA_{A} inverse agonist wherein the inverse agonist has functional potency (EC50 values) at the α1 and/or α5 receptor subtypes of 200 nM, preferably less than 150 nM, and a pharmaceutically acceptable carrier.

This invention also provides a pharmaceutical composition comprising an acetylcholinesterase inhibitor and an inverse agonist of the GABA_{A} α5 receptor wherein the inverse agonist has a functional efficacy at the α5 receptor subtype of less than −5%, preferably less than −10%, and the efficacy measured at the α1, α2 and α3 receptor subtypes is greater than 5% or preferably greater than 10%, and a and a pharmaceutically acceptable carrier.

This invention also provides a pharmaceutical composition comprising an acetylcholinesterase inhibitor and an inverse agonist of the GABA_{A} α5 receptor wherein the inverse agonist has a functional potency (EC50 values) at the α5 receptor subtype of 200 nM, preferably less than 150 nM, and a pharmaceutically acceptable carrier.

This invention also provides a pharmaceutical composition comprising an acetylcholinesterase inhibitor and a GABA_{A} inverse agonist wherein the inverse agonist at the α1 and/or α5 receptor subtypes have a binding Ki of 100 nM, preferably less than 30 nM, and a pharmaceutically acceptable carrier.

This invention also provides a pharmaceutical composition comprising a pharmaceutically acceptable carrier, a GABA_{A} inverse agonist, and an acetylcholinesterase inhibitor, wherein said GABA_{A} inverse agonist is selected from a compound of Formula I below: 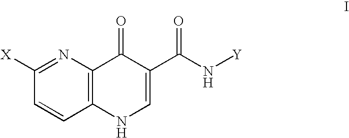
wherein:
X is hydrogen, halogen, —OR₁, NR₂R₃, C₁-C₆ alkyl optionally substituted with up to three groups selected independently from halogen and hydroxy, or —NR₂R₃; or
X is phenyl, naphthyl, 1-(5,6,7,8-tetrahydro)naphthyl or 4-(1,2-dihydro)indenyl, pyridinyl, pyrimidyl, isoquinolinyl, 1,2,3,4-tetrahydroisoquinolinyl, benzofuranyl, benzothienyl, each of which is optionally substituted with up to three groups selected from halogen, C₁-C₆ alkyl, C₁-C₄ alkoxy, C₁-C₆ alkylthio, hydroxy, amino, mono or di(C₁-C₆) alkylamino, cyano, nitro, trifluoromethyl; or
X represents a carbocyclic group (“the X carbocyclic group”) containing from 3-7 members, up to two of which members are optionally hetero atoms selected from oxygen and nitrogen, where the X carbocyclic group is optionally substituted with one or more groups selected from halogen, (C₁-C₆)alkoxy, mono- or di(C₁-C₆)alkylamino, sulfonamide, aza(C₃-C₇)cycloalkyl, (C₃-C₇)cycloalkylthio, (C₁-C₆)alkylthio, phenylthio, or a heterocyclic group; and
Y is lower alkyl having 1-8 carbon atoms optionally substituted with up to two groups selected from halogen, (C₁-C₆)alkoxy, mono- or di(C₁-C₆)alkylamino, sulfonamide, aza(C₃-C₇)cycloalkyl (C₃-C₇)cycloalkylthio, (C₁-C₆)alkylthio, phenylthio, a heterocyclic group, —OR₄, —NR₅R₆, SR₇, or aryl; or
Y is a carbocyclic group (“the Y carbocyclic group”) having from 3-7 members atoms, where up to three of which members are optionally hetero atoms selected from oxygen and nitrogen and where any member of the Y carbocyclic group is optionally substituted with halogen, —OR₄, —NR₅R₆, SR₇, aryl or a heterocyclic group; and
R₁ is hydrogen, lower alkyl having 1-6 carbon atoms, or cycloalkyl having 3-7 carbon atoms, where each alkyl may be optionally substituted with —OR₄ or —NR₅R₆;
R₂ and R₃ are the same or different and represent hydrogen, lower alkyl optionally mono- or disubstituted with alkyl, aryl, halogen, or mono- or di-lower alkyl;
aryl or aryl (C₁-C₆)alkyl where each aryl is optionally substituted with up to three groups selected from halogen, hydroxy, C₁-C₆ alkyl, C₁-C₆ alkoxy, or mono- or di (C₁-C₆)alkylamino;
cycloalkyl having 3-7 carbon atoms optionally mono or disubstituted with halogen, alkoxy, or mono- or di- lower alkyl; or
—SO₂R₈;
R₄ is as defined for R₁;
R₅ and R₆ carry the same definitions as R₂ and R₃, respectively;
R₇ is hydrogen, lower alkyl having 1-6 carbon atoms, or cycloalkyl having 3-7 atoms; and
R₆ is lower alkyl having 1-6 carbon atoms, cycloalkyl having 3-7 carbon atoms, or optionally substituted phenyl,
or a prodrug thereof, or pharmaceutically acceptable salt or solvate of said compound or prodrug,
said composition being effective in the treatment of a cognitive disorder.

This invention also provides a pharmaceutical composition comprising a pharmaceutically acceptable carrier, a GABA_{A} inverse agonist, and an acetylcholinesterase inhibitor, wherein said GABA_{A} inverse agonist is selected from the group consisting of:
N-n-Butyl-6-chloro-4-oxo-1,4-tetrahydro-1,5-naphthyridine-3-carboxamide;
N-n-Butyl-6-ethoxy-4-oxo-1,4-tetrahydro-1,S-naphthyridine-3-carboxamide;
N-(2-Ethylthio)ethyl-6-methoxy-4-oxo-1,4-tetrahydro-1,5-naphthyridine-3-carboxamide;
N-n-Pentyl-6-ethoxy-4-oxo-1,4-tetrahydro-1,5-naphthyridine-3-carboxamide;
N-Benzyl-6-ethoxy-4-oxo-1,4-tetrahydro-1,5-naphthyridine-3-carboxamide;
N-(2-Tetrahydrofuranyl)methyl-6-ethoxy-4-oxo-1,4-tetrahydro-1,5 naphthyridine-3-carboxamide;
N-Isoamyl-6-ethoxy-4-oxo-1,4-tetrahydro-1,5-naphthyridine-3 carboxamide;
N-(3-Methoxybenzyl)-6-ethoxy4-oxo-1,4-tetrahydro-1,5-naphthyridine-3-carboxamide;
N-(3-Ethoxy)propyl-6-ethoxy-4-oxo-1,4-tetrahydro-1,5-naphthyridine-3-carboxamide;
N-2-(2-Methyl)butyl-6-ethoxy-4-oxo-1,4-tetrahydro-1,5-naphthyridine-3 carboxamide;
N-5-Pentanol-6-ethoxy4-oxo-1,4-tetrahydro-1,5-naphthyridine-3-carboxamide;
N-Benzyl-6-methoxy4-oxo-1,4-tetrahydro-1,5-naphthyridine-3-carboxamide;
N-(2-Fluorobenzyl)-6-methoxy4-oxo-1,4-tetrahydro-1,5-naphthyridine-3-carboxamide;
N-(3-Fluorobenzyl)-6-methoxy4-oxo-1,4-tetrahydro-1,5-naphthyridine-3-carboxamide;
N-(4-Fluorobenzyl)-6-methoxy4-oxo-1,4-tetrahydro-1,5-naphthyridine-3-carboxamide;
N-(⅘-Imidazolyl)methyl-6-ethoxy-4-oxo-1,4-tetrahydro-1,5-naphthyridine-3-carboxamide;
N-(3-Thienyl)methyl-6-ethoxy-4-oxo-1,4-tetrahydro-1,5-naphthyridine-3-carboxamide;
N-(2-Tetrahydropyranyl)methyl-6-ethoxy4-oxo-1,4-tetrahydro-1,5-naphthyridine-3-carboxamide;
N-(2-Fluorobenzyl)-6-ethoxy4-oxo-1,4-tetrahydro-1,5-naphthyridine-3-carboxamide;
N-(3,5-Fluorobenzyl)-6-ethoxy4-oxo-1,4-tetrahydro-1,5-naphthyridine-3-carboxamide;
N-(4-Fluorobenzyl)-6-ethoxy4-oxo-1,4-tetrahydro-1,5-naphthyridine-3-carboxamide;
N-(4-Methoxybenzyl)-6-ethoxy-4-oxo-1,4-tetrahydro-1,5-naphthyridine-3-carboxamide;
N-(4-Methylbenzyl)-6-ethoxy-4-oxo-1,4-tetrahydro-1,5-naphthyridine-3-carboxamide;
N-(2-Thienyl)methyl-6-(2-methoxyethoxy)-4-oxo-1,4-tetrahydro-1,5-naphthyridine-3-carboxamide;
N-(2-Thienyl)methyl-6-morpholino4-oxo-1,4-tetrahydro-1,5-naphthyridine-3-carboxamide;
N-(2-Thienyl)methyl-6-dimethylamino4-oxo-1,4-tetrahydro-1,5-naphthyridine-3-carboxamide;
N-(4-Methylaminomethyl)benzyl-6-ethoxy4-oxo-1,4-tetrahydro-1,5-naphthyridine-3-carboxamide;
N-(3-Methylaminomethyl)benzyl-6-ethoxy-4-oxo-1,4-tetrahydro-1,5 naphthyridine-3-carboxamide hydrochloride; and
N-[4-(Imidazolylmethy)lbenzyl-6-ethoxy-4-oxo-1,4-tetrahydro-1,5-naphthyridine-3-carboxamide,
or a prodrug thereof, or pharmaceutically acceptable salt or solvate of said compound or prodrug,
said composition being effective in the treatment of a cognitive disorder.

In a preferred embodiment, the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug.

Non-limiting examples of acetylcholinesterase inhibitors include Aricept (donepezil, E2020), Exelon (rivastigmine), metrifonate, galantamine, physostigmine, tacrine, huperzine A, and icopezil.

In a preferred embodiment, the acetylcholinesterase inhibitor is Aricept (donepezil, E2020), or a prodrug thereof, or a pharmaceutically acceptable salt or solvate of said compound or prodrug.

In a further preferred embodiment, the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug; and the acetylcholinesterase inhibitor is Aricept (donepezil, E2020) or a prodrug thereof, or a pharmaceutically acceptable salt or solvate of said compound or prodrug.

This invention also provides a pharmaceutical composition comprising a pharmaceutically acceptable carrier, a GABA_{A} inverse agonist and an acetylcholinesterase inhibitor, wherein said GABA_{A} inverse agonist compound is selected from a compound which is wherein
A is C₁-C₆ alkylene;
R_{d} and Rₑ are independently lower alkyl groups,
or a prodrug thereof, or a pharmaceutically acceptable salt or solvate of said compound or prodrug,
said composition being effective in the treatment of a cognitive disorder.

This invention also provides a pharmaceutical composition comprising a pharmaceutically acceptable carrier, a GABA_{A} inverse agonist, and an acetylcholinesterase inhibitor wherein said GABA_{A} inverse agonist is selected from a compound which is 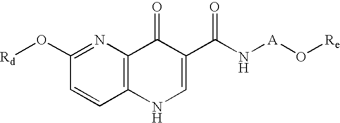
wherein
A is C₁-C₆ alkylene;
R_{d} is lower alkyl; and
R_{f} is a group of the formula: 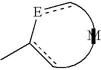
   where E is oxygen or nitrogen; and
M is C₁-C₃ alkylene or nitrogen,
or a prodrug thereof, or a pharmaceutically acceptable salt or solvate of said compound or prodrug,
said composition being effective in the treatment of a cognitive disorder.

This invention also provides a pharmaceutical composition comprising a pharmaceutically acceptable carrier, a GABA_{A} inverse agonist, and an acetylcholinesterase inhibitor, wherein said GABA_{A} inverse agonist is selected from a compound which is 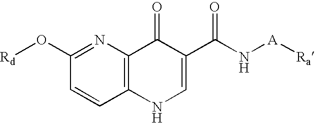
wherein
A is C₁-C₆ alkylene;
R_{d} is lower alkyl; and
Rₐ′ is phenyl optionally mono-, di- or trisubstituted with halogen, lower alkyl, lower alkoxy, or mono- or di-C₁-C₆ alkylamino, or mono-di-C₁-C₆ alkylamino lower alkyl; or
Rₐ′ is a heteroaryl group, that is, one or more aromatic ring systems of 5-,6- or 7-membered rings containing at least one and up to four hetero atoms selected from nitrogen, oxygen or sulfur, said composition being effective in the treatment of a cognitive disorder,
or a prodrug thereof, or a pharmaceutically acceptable salt or solvate of said compound or prodrug,
said composition being effective in the treatment of a cognitive disorder.

Heteroaryl groups include, for example, thienyl, furanyl, thiazolyl, imidazolyl, (is)oxazolyl, pyridyl, pyrimidinyl, (iso)quinolinyl, naphthyridinyl, benzimidazolyl, and benzoxazolyl.

This invention also provides a pharmaceutical composition comprising a pharmaceutically acceptable carrier, a GABA_{A} inverse agonist, and an acetylcholinesterase inhibitor, wherein said GABA_{A} inverse agonist compound is selected from a compound which is 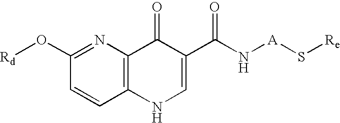
wherein
A is C₁-C₆ alkylene; and
R_{d} and Rₑ are independently lower alkyl groups,
or a prodrug thereof, or a pharmaceutically acceptable salt or solvate of said compound or prodrug,
said composition being effective in the treatment of a cognitive disorder.

This invention also provides a pharmaceutical composition comprising a pharmaceutically acceptable carrier, a GABA_{A} inverse agonist, and an acetylcholinesterase inhibitor, wherein said GABA_{A} inverse agonist is selected from a compound which is 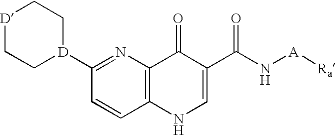
wherein
D is nitrogen or CH;
D′ is nitrogen or oxygen;
A is C₁-C₆ alkylene; and
Rₐ′ is phenyl optionally mono-, di- or trisubstituted with halogen, lower alkyl, lower alkoxy, or mono- or di-C₁-C₆ alkylamino, or mono- or di-C₁-C₆ alkylamino lower alkyl,
or a prodrug thereof, or a pharmaceutically acceptable salt or solvate of said compound or prodrug,
said composition being effective in the treatment of a cognitive disorder.

This invention also provides a pharmaceutical composition comprising a pharmaceutically acceptable carrier, a GABA_{A} inverse agonist, and an acetylcholinesterase inhibitor, wherein said GABA_{A} inverse agonist is selected from a compound which is 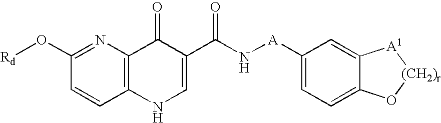
wherein
A is C₁-C₆ alkylene; and
R_{d} is lower alkyl;
A′ represents oxygen or methylene; and
r is an integer of from 1-3,
or a prodrug thereof, or a pharmaceutically acceptable salt or solvate of said compound or prodrug,
said composition being effective in the treatment of a cognitive disorder.

This invention also provides a pharmaceutical composition comprising a pharmaceutically acceptable carrier, a GABA_{A} inverse agonist, and an acetylcholinesterase inhibitor, wherein said GABA_{A} inverse agonist is selected from a compound which is 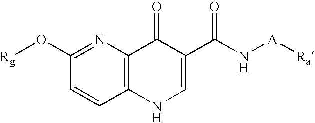
wherein
A is C₁-C₆ alkylene;
R_{g} is lower alkyloxy lower alkyl; and
Rₐ′ is phenyl optionally mono-, di-, or trisubstituted with halogen, lower alkyl, lower alkoxy, or mono- or di-C₁-C₆ alkylamino, or mono- or di-C₁-C₆ alkylamino lower alkyl,
or a prodrug thereof, or a pharmaceutically acceptable salt or solvate of said compound or prodrug,
said composition being effective in the treatment of a cognitive disorder.

This invention also provides a pharmaceutical composition comprising a pharmaceutically acceptable carrier, a GABA_{A} inverse agonist, and an acetylcholinesterase inhibitor, wherein said GABA_{A} inverse agonist is selected from a compound which is 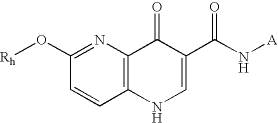
wherein
A is lower alkyl having 1-8 carbon atoms or cycloalkyl having 3-7 carbon atoms, any of which may be optionally substituted with one or more hydroxy groups and Rₕ is lower alkyl,
or a prodrug thereof, or a pharmaceutically acceptable salt or solvate of said compound or prodrug,
said composition being effective in the treatment of a cognitive disorder.

The pharmaceutical compositions of the present invention are useful for treating cognitive disorders in a mammal. Non-limiting examples of such cognitive disorders include Alzheimer's disease, mild cognitive impairment, age-related cognitive decline, vascular dementia, Parkinson's disease, memory impairment associated with depression or anxiety, psychosis, Down's Syndrome, stroke, traumatic brain injury, and attention deficit disorder.

In a preferred embodiment, the cognitive disorder is Alzheimer's Disease.

In another preferred embodiment, the cognitive disorder is mild cognitive impairment.

This invention also provides a method for treating a cognitive disorder in a mammal, comprising administering to a mammal in need of such treatment an effective amount of a combination of a GABA_{A} inverse agonist and an acetylcholinesterase inhibitor. As used herein, a “combination” of a GABA_{A} inverse agonist and an acetylcholinesterase inhibitor is obtained when the GABA_{A} inverse agonist and the acetylcholinesterase inhibitor are administered separately, sequentially or simultaneously, where the benefit of the combination is obtained. When the GABA_{A} inverse agonist and the acetylcholinesterase inhibitor are administered simultaneously, they may be administered either in the same pharmaceutical composition or in different pharmaceutical compositions.

In a preferred embodiment, the acetylcholinesterase inhibitor is Aricept (donepezil, E2020) or a prodrug thereof, or a pharmaceutically acceptable salt or solvate of said compound or prodrug.

In a further preferred embodiment, the GABA_{A} inverse agonist is N-Benzyl-6-ethoxy4-oxo-1,4-tetrahydro-1,5-naphthyridine-3-carboxamide, or a prodrug thereof, or a pharmaceutically acceptable salt or solvate of said compound or prodrug.

In a further preferred embodiment, the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug; and the acetylcholinesterase inhibitor is Aricept (donepezil, E2020) or a prodrug thereof, or a pharmaceutically acceptable salt or solvate of said compound or prodrug.

As used herein, the benefit of the combination treatment is obtained where treatment with a combination of a GABA_{A} cognitive enhancer and an AChE inhibitor results in greater (either additive or synergistic) efficacy or cognitive/behavioral improvement in the treatment of a cognitive disorder, such as any of the above listed disorders, in comparison to the efficacy displayed by either agent alone. Such a combination preferably allows lower doses of each agent to be administered, resulting in efficacy similar to or greater than that observed with higher doses of either agent alone, and with reduced side effects (or higher therapeutic index). In a preferred embodiment, the combination treatment provides a synergistic therapeutic effect. In another preferred embodiment the combination treatment provides at least an additive effect with reduced side effects.

As used herein, a mammal in need of treatment of a cognitive disorder means a mammal, and preferably a human, that is suffering from, or is at risk of suffering from, a cognitive disorder.

As used herein, the terms “treat”, “treating” and “treatment”, and the like, as applied to cognitive disorders, refer to methods that slow, ameliorate, reduce or reverse such a disorder or any symptoms associated with said disorder, as currently afflicting the subject, as well as methods that prevent such a disorder or any symptoms thereof, from occurring.

The present invention further provides the use of a GABA_{A} inverse agonist and an acetylcholinesterase inhibitor in the manufacture of a medicament for treating a cognitive disorder. The GABA_{A} inverse agonist and an acetylcholinesterase inhibitor may be combined in a single medicament or maintained in separate medicaments.

Non-limiting examples of acetylcholinesterase inhibitors include Aricept (donepezil, E2020), Exelon (rivastigmine), metrifonate, galantamine, physostigmine, tacrine, huperzine A, and icopezil.

In a preferred embodiment, the acetylcholinesterase inhibitor is Aricept (donepezil, 2020) or a prodrug thereof, or a pharmaceutically acceptable salt or solvate of said compound or prodrug.

In a further preferred embodiment, the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug.

In a further preferred embodiment, the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug; and the acetylcholinesterase inhibitor is Aricept (donepezil, E2020) or a prodrug thereof, or a pharmaceutically acceptable salt or solvate of said compound or prodrug.

The present invention also provides a kit comprising:
a) a first compound being a GABA_{A} inverse agonist as described above, and most preferably a compound of formula I, or an isomer thereof, a prodrug of said compound or isomer, or a pharmaceutically acceptable salt or solvate of said compound, isomer or prodrug; and a pharmaceutically acceptable carrier, vehicle or diluent in a first unit dosage form;
b) a second compound selected from the group consisting of an acetylcholinesterase inhibitor; and a pharmaceutically acceptable carrier, vehicle or diluent in a second unit dosage form; and
c) a container for containing said first and second unit dosage forms wherein the amounts of said first and second compounds result in an enhanced therapeutic effect, as described above.

The kit may further comprise a printed label or a set of printed instructions directing the use of the pharmaceutical composition to treat a cognitive disorder.

### BRIEF DESCRIPTION OF THE DRAWING

FIG. 1 graphically demonstrates that non-effective doses of Aricept and a compound of Formula I when co-administered interact to attenuate scopolamine-induced deficits in the spatial water maze (see text for details).

### BRIEF DESCRIPTION OF THE INVENTION

The GABA_{A} ligands disclosed above may be prepared by the methods described in PCT publication WO 99/10347 by Neurogen Corporation, published Mar. 4, 1999, which is incorporated herein by reference.

By lower alkyl in the present invention is meant straight or branched chain alkyl groups having 1-6 carbon atoms, such as, for example, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

By cycloalkyl in the present invention is meant cycloalkyl groups having 3-7 atoms, such as, for example, cyclopropyl, cyclobutyl, cyclopentyl, cyclohexyl, and cycloheptyl.

By aryl is meant an aromatic carbocyclic group having a single ring (e.g., phenyl), multiple rings (e.g., biphenyl), or multiple condensed rings in which at least one is aromatic, (e.g., 1,2,3,4-tetrahydronapthyl, naphthyl, anthryl, or phenanthryl), which is optionally mono-, di-, or trisubsituted with, e.g., halogen, lower alkyl, lower alkoxy, lower alkylthio, trifluoromethyl, lower acyloxy, aryl, heteroaryl, and hydroxy.

By lower alkoxy in the present invention is meant straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

By cycloalkoxy in the present invention is meant cycloalkylalkoxy groups having 3-7 carbon atoms where cycloalkyl is defined above.

By halogen in the present invention is meant fluorine, bromine, chlorine, and iodine.

By heteroaryl (aromatic heterocycle) in the present invention is meant one or more aromatic ring systems of 5-, 6-, or 7-membered rings containing at least one and up to four hetero atoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, naphthridinyl, benzimidazolyl, and benzoxazolyl.

In certain situations, GABA_{A} inverse agonists useful according to the present invention may contain one or more asymmetric carbon atoms, so that the compounds can exist in different stereoisomeric forms. These compounds can be, for example, racemates or optically active forms. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent, or chromatography, using for example a chiral HPLC column.

Representative compounds useful in the combination of the present invention include those compounds described above, and their pharmaceutically acceptable acid and base addition salts and solvates thereof. If the compound of the invention is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds.

Non-toxic pharmaceutical salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)n-COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

The present invention also encompasses the use of prodrugs of either or both of the active compounds used in the combination therapy of the present invention. For example, those skilled in the art will recognize various synthetic methodologies which may be employed to prepare pharmaceutically acceptable acylated prodrugs of these compounds. Additional types of prodrugs are also encompassed. For instance, free carboxyl groups of compounds can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The pharmaceutical utility of compounds and compositions of this invention is indicated by the following assays for GABA_{A} receptor activity.

Assays are carried out as described in Thomas and Tallman (J. Bio. Chem. 156: 9838-9842, J. Neurosci. 3: 433-440, 1983). Rat cortical tissue is dissected and homogenized in 25 volumes (w/v) of 0.05 M Tris HCl buffer (pH 7.4 at 4° C.). The tissue homogenate is centrifuged in the cold (4° C.) at 20,000×g for 20 min. The supernatant is decanted and the pellet is rehomogenized in the same volume of buffer and again centrifuged at 20,000×g. The supernatant is decanted and the pellet is frozen at −20° C. overnight. The pellet is then thawed and rehomogenized in 25 volume (original wt/vol) of buffer and the procedure is carried out twice. The pellet is finally resuspended in 50 volumes (w/vol of 0.05 M Tris HCl buffer (pH 7.4 at 40° C.).

Incubations contain 100 ml of tissue homogenate, 100 ml of radioligand 0.5 nM (³H-Ro15-1788 [³H-Flumazenil] specific activity 80 Ci/mmol), drug or blocker and buffer to a total volume of 500 ml. Incubations are carried out for 30 minutes at 4° C. then are rapidly filtered through GFB filters to separate free and bound ligand. Filters are washed twice with fresh 0.05 M Tris HCl buffer (pH 7.4 at 4° C.) and counted in a liquid scintillation counter. 1.0 mM diazepam is added to some tubes to determine nonspecific binding. Data are collected in triplicate determinations, averaged and % inhibition of total specific binding is calculated. Total Specific Binding=Total−Nonspecific. In some cases, the amounts of unlabeled drugs are varied and total displacement curves of binding are carried out. Data are converted to Ki's. Compounds of the invention when tested in the assay described above have Ki's of less than 1μM.

In addition, the following assay may be used to determine if the compounds of the invention are agonists, antagonists, or inverse agonists, and, therefore, their specific pharmaceutical utility. The following assay can be employed to determine specific GABA_{A} receptor activity.

Assays are carried out as described in White and Gurley (NeuroReport 6: 1313-1316, 1995) and White, Gurley, Hartnett, Stirling, and Gregory (Receptors and Channels 3: 1-5, 1995) with modifications. Xenopus laevis oocytes are enzymatically isolated and injected with non-polyadenylated cRNA mixed in a ratio of 4:1:4 for human derived α, β, and γ subunits, respectively. For each subunit combination, sufficient message is injected to result in current amplitudes of >10 nA when 1 μM GABA is applied.

Electrophysiological recordings are carried out using the two electrode voltage-clamp technique at a membrane holding potential of −70 mV.

Compounds are evaluated against a GABA concentration that evokes <10% of the maximal evokable GABA current. Each oocyte is exposed to increasing concentrations of compound in order to evaluate a concentration/effect relationship. Compound efficacy is expressed as a percent-change in current amplitude: 100* ((Ic/I)−1), where Ic is the GABA evoked current amplitude observed in the presence of compound and I is the GABA evoked current amplitude observed in the absence of compound.

Specificity of a compound for the Ro15-1788 site is determined following completion of the concentration/effect curve. After washing the oocyte sufficiently to remove previously applied compound, the oocyte is exposed to GABA+1μM Ro15-1788, followed by exposure to GABA+1 μM Ro15-1788+compound. Percent change due to addition of compound is calculated as described above. Any percent change observed in the presence of Ro15-1788 is subtracted from the percent changes in current amplitude observed in the absence of 1 μM Ro15-1788. These net values are used for the calculation of average efficacy and EC₅₀ values.

To evaluate average efficacy and EC₅₀ values, the concentration/effect data are averaged across cells and fit to the logistic equation. Average values are reported as mean ± standard error.

The compositions of this invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. One or more compounds of this invention may be present in association with one or more non-toxic pharmaceutically acceptable carriers and/or diluents and/or adjuvants and if desired other active ingredients. The pharmaceutical compositions containing compounds of this invention may be suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredients in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example corn starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyphenylene sorbitol monooleate. The aqueous suspension may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example, soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of this invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compounds of this invention may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Administration of the compositions of this invention can be via any method which delivers a compound of this invention systemically and/or locally. These methods include oral routes and transdermal routes, etc. Generally, the compounds of this invention are administered orally, but parenteral administration may be utilized (e.g., intravenous, intramuscular, subcutaneous or intramedullary). The two different compounds of this invention can be co-administered simultaneously or sequentially in any order, or a single pharmaceutical composition comprising both a GABA_{A} inverse agonist as described above and an acetylcholinesterase inhibitor as described above in a pharmaceutically acceptable carrier can be administered.

The amount and timing of compounds administered will, of course, be based on the judgment of the prescribing physician. Thus, because of patient-to-patient variability, the dosages given below are a guideline and the physician may titrate doses of the agent to achieve the activity that the physician considers appropriated for the individual patient. In considering the degree of activity desired, the physician must balance a variety of factors such as cognitive function, age of the patient, presence of preexisting disease, as well as presence of other disease (e.g., cardiovascular). The following paragraphs provide preferred dosage ranges for the various components of this invention (based on average human weight of 70 kg).

In general, an effective dosage for the GABA_{A} is in the range of 0.001 to 30 mg/kg/day, preferably 0.01 to 10.0 mg/kg/day.

In general an effective dosage for the acetylcholinesterase inhibitor is in the range of 0.01 to 10 mg/kg/day. More specific dosages are as follows:

The specific dosages for the cholinesterase/butylcholinesterase inhibitors are as follows:
For donepezil (Aricept™) the range is 0.01 to 0.75 mg/kg/day.
For tacrine (Cognex™) the range is 0.1 to 2.3 mg/kg/day.
For rivastigmine (Exelon™) the range is 0.1 to 0.5 mg/kg/day.
For physostigmine (Synapton) the range is 0.01 to 0.4 mg/kg/day.
For galantamine (Reminyl) the range is 0.05 to 1.0 mg/kg/day.
For metrifonate (Promem) the range is 0.1 to 2.0 mg/kg/day.

It will be understood, however, that the specific dose level for any particular patient will depend up on a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It will be convenient to formulate these animal feed and drinking water compositions with a mullet-dose of the drug so that the animal takes in an appropriate quantity of the composition along with its diet. It will also be convenient to present the composition as a premix for addition to the feed or drinking water.

### EXAMPLE 1

The following experiment demonstrates that sub-efficacious doses of Aricept and a compound of Formula I when used in combination attenuate a scopolamine-induced memory deficit in the spatial water maze task.

Method

Subjects: Animals used in these studies were naive male Sprague Dawley rats (SASCO St. Louis) weighing between 200-250 grams. Animals were housed in groups of three in a temperature (22° C.±2° C.) and humidity (40-70% relative humidity) controlled vivarium with a 12-hour light/dark cycle. Animals had ad lib access to food and water.

Drugs: Aricept and said compound of Formula I were each dissolved in 50% polyethylene glycol (PEG), and scopolamine HC1 (Sigma) was dissolved in 0.9% saline. Aricept and said compound of Formula I (alone or in combination) or 50% PEG was administered intravenously (IV) 5 minutes prior to scopolamine (0.125 mg/kg) or saline given intraperitoneally (IP). Training commenced 15 minutes after the IP injection.

Apparatus: The water maze apparatus consists of a circular tank (120 cm in diameter and 56 cm in height) with a black interior. The tank was filled with water (23° C.) to a height of approximately 40 cm. Superimposed onto the tank were four quadrants (North, South, East and West). The tank was surrounded by external visual cues that consisted of a black and white checkered wall, a black and white striped wall, a blue wall, and a white wall. A stationary black circular Plexiglass platform with a black neoprene rubber top was placed in the northeast quadrant approximately 1 cm below the surface of the water.

Procedure: An animal was initially placed on the platform in the tank for 20 seconds. Thereafter, the 6 trial acquisition training was begun by placing the rat in the water at the South entry position. The trial ended with the animal finding the platform or being placed onto it after 90 sec. Each of the subsequent five training trials was separated by an intertrial interval (ITI) of 2 minutes and was begun by placing the rat at different entry positions, the order of which was pseudo-randomized. One day after training, each drug-free animal was individually tested for retention on one trial. For each trial during acquisition and retention, a computerized video tracking system recorded the latency (sec) to reach the submerged platform, the total distance traveled (m) in the water maze, the number of zone (quadrant) transitions made, and the swim speed of the animal.

Data Analysis: A one-way ANOVA was conducted on the latency to locate the platform during retention testing. Tests for significant differences between individual treatment groups were assessed using a Fisher LSD test (p<0.05).

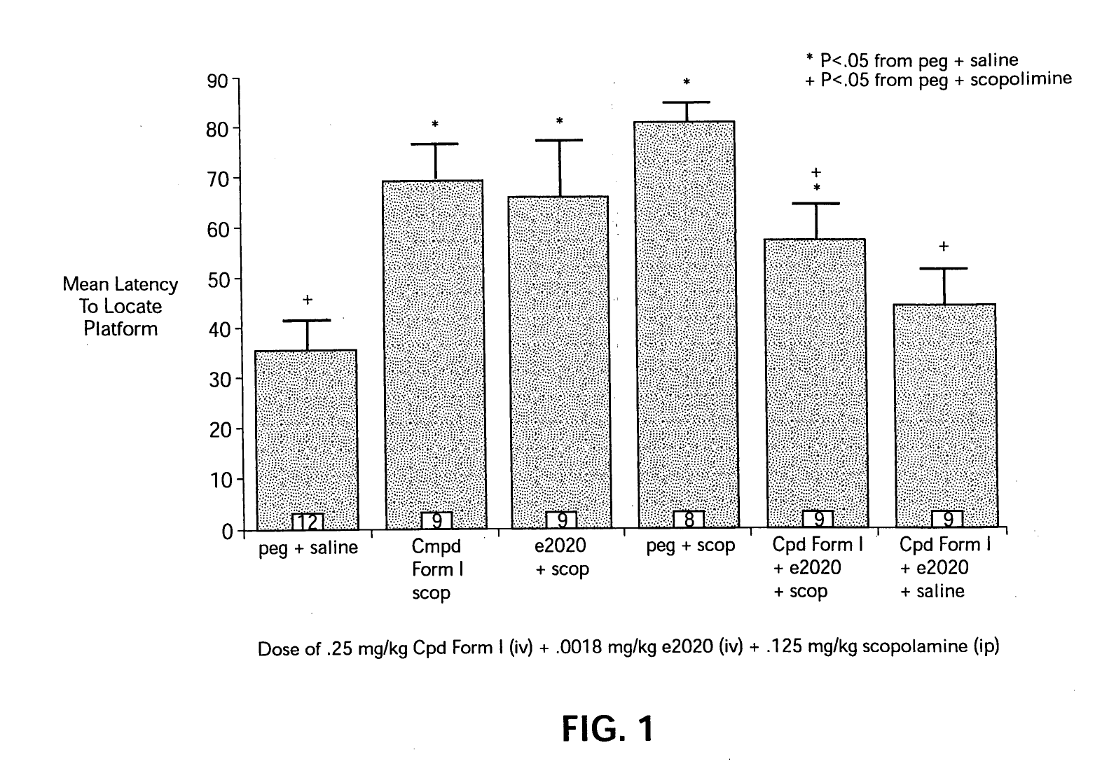

Results and Discussion: An ANOVA conducted on the latency to locate the platform during retention testing revealed a significant overall effect of treatment [F (5,50)=5.92, p<.01], and was followed up by comparisons between individual groups using the Fisher's LSD test (FIG. 1). Animals treated with PEG/scopolamine showed a longer latency to find the platform signifying a retention deficit compared to animals treated with PEG/saline. As expected with the chosen doses, Aricept and the compound of Formula I, when administered alone, did not significantly attenuate the impairing effects of scopolamine in this task. However, co-administration of Aricept and the compound of Formula I resulted in a statistically significant attenuation of a scopolamine induced retention deficit. Thus, animals receiving Aricept/compound of Formula I/scopolamine prior to acquisition training found the platform in a significantly shorter time compared to animals treated with PEG/scopolamine during retention testing.

These results demonstrate that non-effective doses of Aricept and a compound of Formula I, when co-administered, interact to attenuate scopolamine-induced deficits in the spatial water maze. These findings show the benefit of combining the two drugs to enhance memory.

All patents, patent applications, and publications cited above are incorporated herein by reference in their entirety.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description. Such modifications are intended to fall within the scope of the appended claims.

1. A pharmaceutical composition comprising an acetylcholinesterase inhibitor and an inverse agonist of the GABA_{A} α1 and/or α5 receptor subtype wherein the inverse agonist has a functional efficacy at the α1 and/or α5 receptor subtypes of less than −5%, preferably less than −10%, and the efficacy measured at the α2 and α3 receptor subtypes is greater than 5% or preferably greater than 10%, and a pharmaceutically acceptable carrier, said composition being effective in the treatment of a cognitive disorder.

2. The pharmaceutical composition of claim 1, wherein the inverse agonist has a functional potency (EC50 values) at the α1 and/or α5 receptor subtypes of 200 nM, preferably less than 150 nM.

3. The pharmaceutical composition of claim 1, wherein the inverse agonist has a functional efficacy at the α5 receptor subtype of less than −5%, preferably less than −10%, and the efficacy measured at the α1, α2 and α3 receptor subtypes is greater than 5% or preferably greater than 10%.

4. The pharmaceutical composition of claim 3, wherein the inverse agonist has a functional potency (EC50 values) at the α5 receptor subtype of 200 nM, preferably less than 150 nM.

5. The pharmaceutical composition of claim 1, wherein the inverse agonist at the α1 and/or α5 receptor subtypes has a binding Ki of 100 nM, preferably less than 30 nM.

6. A pharmaceutical composition comprising a pharmaceutically acceptable carrier, a GABA_{A} inverse agonist, and an acetylcholinesterase inhibitor, wherein said GABA_{A} inverse agonist is selected from a compound of Formula I below: 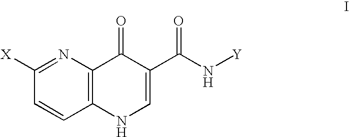
wherein:
X is hydrogen, halogen, —OR₁, NR₂R₃, C₁-C₆ alkyl optionally substituted with up to three groups selected independently from halogen and hydroxy, or —NR₂R₃; or
X is phenyl, naphthyl, 1-(5,6,7,8-tetrahydro)naphthyl or 4-(1,2-dihydro)indenyl, pyridinyl, pyrimidyl, isoquinolinyl, 1,2,3,4-tetrahydroisoquinolinyl, benzofuranyl, benzothienyl, each of which is optionally substituted with up to three groups selected from halogen, C₁-C₆ alkyl, C₁-C₄ alkoxy, C₁-C₆ alkylthio, hydroxy, amino, mono or di(C₁-C₆) alkylamino, cyano, nitro, trifluoromethyl; or
X represents a carbocyclic group (“the X carbocyclic group”) containing from 3-7 members, up to two of which members are optionally hetero atoms selected from oxygen and nitrogen, where the X carbocyclic group is optionally substituted with one or more groups selected from halogen, (C₁-C₆)alkoxy, mono- or di(C₁-C₆)alkylamino, sulfonamide, aza(C₃-C₇)cycloalkyl, (C₃-C₇)cycloalkylthio, (C₁-C₆)alkylthio, phenylthio, or a heterocyclic group; and
Y is lower alkyl having 1-8 carbon atoms optionally substituted with up to two groups selected from halogen, (C₁-C₆)alkoxy, mono- or di(C₁-C₆)alkylamino, sulfonamide, aza(C₃-C₇)cycloalkyl (C₃-C₇)cycloalkylthio, (C₁-C₆)alkylthio, phenylthio, a heterocyclic group, —OR₄, —NR₅R₆, SR₇, or aryl; or
Y is a carbocyclic group (“the Y carbocyclic group”) having from 3-7 members atoms, where up to three of which members are optionally hetero atoms selected from oxygen and nitrogen and where any member of the Y carbocyclic group is optionally substituted with halogen, —OR₄, —NR₅R₆, SR₇, aryl or a heterocyclic group; and
R₁ is hydrogen, lower alkyl having 1-6 carbon atoms, or cycloalkyl having 3-7 carbon atoms, where each alkyl may be optionally substituted with —OR₄ or
—NR₅R₆;
R₂ and R₃ are the same or different and represent hydrogen, lower alkyl optionally mono- or disubstituted with alkyl, aryl, halogen, or mono- or di-lower alkyl;
aryl or aryl (C₁-C₆)alkyl where each aryl is optionally substituted with up to three groups selected from halogen, hydroxy, C₁-C₆ alkyl, C₁-C₆ alkoxy, or mono- or di (C₁-C₆)alkylamino;
cycloalkyl having 3-7 carbon atoms optionally mono or disubstituted with halogen, alkoxy , or mono- or di- lower alkyl; or
—SO₂R₈;
R₄ is as defined for R₁;
R₅ and R₆ carry the same definitions as R₂ and R₃, respectively;
R₇ is hydrogen, lower alkyl having 1-6 carbon atoms, or cycloalkyl having 3-7 atoms; and
R₈ is lower alkyl having 1-6 carbon atoms, cycloalkyl having 3-7 carbon atoms, or optionally substituted phenyl,
or a pharmaceutically acceptable prodrug thereof, or a pharmaceutically acceptable salt or solvate of said compound or prodrug,
said composition being effective in the treatment of a cognitive disorder.

7. A pharmaceutical composition comprising a pharmaceutically acceptable carrier, a GABA_{A} inverse agonist, and an acetylcholinesterase inhibitor, wherein the GABA_{A} inverse agonist is selected from the group consisting of:
N-n-Butyl-6-chloro-4-oxo-1,4-tetrahydro-1,5-naphthyridine-3-carboxamide;
N-n-Butyl-6-ethoxy4-oxo-1,4-tetrahydro-1,S-naphthyridine-3-carboxamide;
N-(2-Ethylthio)ethyl-6-methoxy4-oxo-1,4-tetrahydro-1,5-naphthyridine-3-carboxamide;
N-n-Pentyl-6-ethoxy-4-oxo-1,4-tetrahydro-1,5-naphthyridine-3-carboxamide;
N-Benzyl-6-ethoxy-4-oxo-1,4-tetrahydro-1,5-naphthyridine-3-carboxamide;
N-(2-Tetrahydrofuranyl)methyl-6-ethoxy4-oxo-1,4-tetrahydro-1,5 naphthyridine-3-carboxamide;
N-Isoamyl-6-ethoxy-4-oxo-1,4-tetrahydro-1,5-naphthyridine-3 carboxamide;
N-(3-Methoxybenzyl)-6-ethoxy4-oxo-1,4-tetrahydro-1,5-naphthyridine-3-carboxamide;
N-(3-Ethoxy)propyl-6-ethoxy-4-oxo-1,4-tetrahydro-1,5-naphthyridine-3-carboxamide;
N-2-(2-Methyl)butyl-6-ethoxy-4-oxo-1,4-tetrahydro-1,5-naphthyridine-3 carboxamide;
N-5-Pentanol-6-ethoxy4-oxo-1,4-tetrahydro-1,5-naphthyridine-3-carboxamide;
N-Benzyl-6-methoxy4-oxo-1,4-tetrahydro-1,5-naphthyridine-3-carboxamide;
N-(2-Fluorobenzyl)-6-methoxy4-oxo-1,4-tetrahydro-1,5-naphthyridine-3-carboxamide;
N-(3-Fluorobenzyl)-6-methoxy4-oxo-1,4-tetrahydro-1,5-naphthyridine-3-carboxamide;
N-(4-Fluorobenzyl)-6-methoxy4-oxo-1,4-tetrahydro-1,5-naphthyridine-3-carboxamide;
N-(⅘-Imidazolyl)methyl-6-ethoxy-4-oxo-1,4-tetrahydro-1,5-naphthyridine-3-carboxamide;
N-(3-Thienyl)methyl-6-ethoxy-4-oxo-1,4-tetrahydro-1,5-naphthyridine-3-carboxamide;
N-(2-Tetrahydropyranyl)methyl-6-ethoxy4-oxo-1,4-tetrahydro-1,5-naphthyridine-3-carboxamide;
N-(2-Fluorobenzyl)-6-ethoxy4-oxo-1,4-tetrahydro-1,5-naphthyridine-3-carboxamide;
N-(3,5-Fluorobenzyl)-6-ethoxy4-oxo-1,4-tetrahydro-1,5-naphthyridine-3-carboxamide;
N-(4-Fluorobenzyl)-6-ethoxy4-oxo-1,4-tetrahydro-1,5-naphthyridine-3-carboxamide;
N-(4-Methoxybenzyl)-6-ethoxy4-oxo-1,4-tetrahydro-1,5-naphthyridine-3-carboxamide;
N-(4-Methylbenzyl)-6-ethoxy-4-oxo-1,4-tetrahydro-1,5-naphthyridine-3-carboxamide;
N-(2-Thienyl)methyl-6-(2-methoxyethoxy)4-oxo-1,4-tetrahydro-1,5-naphthyridine-3-carboxamide;
N-(2-Thienyl)methyl-6-morpholino4-oxo-1,4-tetrahydro-1,5-naphthyridine-3-carboxamide;
N-(2-Thienyl)methyl-6-dimethylamino4-oxo-1,4-tetrahydro-1,5-naphthyridine-3-carboxamide;
N-(4-Methylaminomethyl)benzyl-6-ethoxy4-oxo-1,4-tetrahydro-1,5-naphthyridine-3-carboxamide;
N-(3-Methylaminomethyl)benzyl-6-ethoxy4-oxo-1,4-tetrahydro-1,5 naphthyridine-3-carboxamide hydrochloride;
N-[4-(Imidazolylmethy)lbenzyl-6-ethoxy-4-oxo-1,4-tetrahydro-1,5-naphthyridine-3-carboxamide,
a pharmaceutically acceptable prodrug thereof, and a pharmaceutically acceptable salt or solvate of said compound or prodrug,
said composition being effective in the treatment of a cognitive disorder.

8. The pharmaceutical composition of claim 7, wherein the GABA_{A} inverse agonist is N-Benzyl-6-ethoxy4-oxo-1,4-tetrahydro-1,5-naphthyridine-3-carboxamide, or a prodrug thereof, or a pharmaceutically acceptable salt or solvate of said compound or prodrug.

9. The pharmaceutical composition of claim 7, wherein the acetylcholinesterase inhibitor is selected from the group consisting of Aricept (donepezil, E2020), Exelon (rivastigmine), metrifonate, galantamine, physostigmine, tacrine, huperzine A, and icopezil, a prodrug thereof, and a pharmaceutically acceptable salt or solvate of said compound or prodrug.

10. The pharmaceutical composition of claim 9, wherein the acetylcholinesterase inhibitor is Aricept (donepezil, E2020) or a prodrug thereof, or a pharmaceutically acceptable salt or solvate of said compound or prodrug.

11. The pharmaceutical composition of claim 7, wherein the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug; and the acetylcholinesterase inhibitor is Aricept (donepezil, E2020) or a prodrug thereof, or a pharmaceutically acceptable salt or solvate of said compound or prodrug.

12. A method for treating a cognitive disorder in a mammal, comprising administering to a mammal in need of such treatment an effective amount of a combination of a GABA_{A} inverse agonist and an acetylcholinesterase inhibitor, wherein the GABA_{A} inverse agonist and the acetylcholinesterase inhibitor are as defined in claim 1.

13. The method of claim 12, wherein the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug.

14. The method of claim 12, wherein the acetylcholinesterase inhibitor is selected from the group consisting of Aricept (donepezil, E2020), Exelon (rivastigmine), metrifonate, galantamine, physostigmine, tacrine, huperzine A, and icopezil, a prodrug thereof, and a pharmaceutically acceptable salt or solvate of said compound or prodrug.

15. The method of claim 12, wherein the acetylcholinesterase inhibitor is Aricept (donepezil, E2020) or a prodrug thereof, or a pharmaceutically acceptable salt or solvate of said compound or prodrug.

16. The method of claim 12, wherein the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug; and the acetylicholinesterase inhibitor is Aricept (donepezil, E2020) or a prodrug thereof, or a pharmaceutically acceptable salt or solvate of said compound or prodrug.

17. The method of claim 12, wherein the GABA_{A} inverse agonist and the acetylcholinesterase inhibitor are administered separately.

18. The method of claim 12, wherein the GABA_{A} inverse agonist and the acetylcholinesterase inhibitor are administered sequentially.

19. The method of claim 12, wherein the GABA_{A} inverse agonist and the acetylcholinesterase inhibitor are administered simultaneously.

20. The method of claim 12, wherein the cognitive disorder is selected from the group consisting of Alzheimer's disease, mild cognitive impairment, age-related cognitive decline, vascular dementia, Parkinson's disease, memory impairment associated with depression or anxiety, psychosis, Down's Syndrome, stroke, traumatic brain injury, and attention deficit disorder.

21. The method of claim 20, wherein the cognitive disorder is Alzheimer's Disease.

22. The method of claim 20, wherein the cognitive disorder is mild cognitive impairment.
